(19) United States
(12) Patent Application Publication
(10) Pub. No.: US 2004/0101540 A1
(43) Pub. Date: May 27, 2004

(54) Oral delivery system and method for making same

(76) Inventor: John Cooker, Rohrersville, MD (US)

Correspondence Address: LAW OFFICES OF RAYMOND A. NUZZO, LLC, 579 THOMPSON AVENUE, EAST HAVEN, CT, 06512, US

(21) Appl. No.: 10/715,915

(22) Filed: Nov. 18, 2003

Related U.S. Application Data
(63) Continuation-in-part of application No. 09/614,243, filed on Jul. 12, 2000, now Pat. No. 6,656,501.
(60) Provisional application No. 60/151,901, filed on Sep. 1, 1999.

(51) Int. Cl. A61K 9/00; A61K 9/48

(52) U.S. Cl. 424/400; 424/452; 424/451

(57) ABSTRACT

The present invention is directed to improved oral dosage forms that are significantly easier to swallow. In accordance with the present invention, the oral dosage forms are configured to have relatively greater weight and/or density to effect partial or total submergence in the liquid with which the oral dosage form is taken. In one embodiment, the oral dosage form has an active ingredient, an inactive ingredient, and a digestible substance that has a predetermined weight that effects at least partial sinking of the oral dosage form in the liquid. In a preferred embodiment, the active ingredient and inactive ingredient have a combined total weight that is substantially less than the predetermined weight of the digestible substance.

### CROSS-REFERENCE TO RELATED APPLICATIONS

This application is a continuation-in-part and claims the benefit of commonly owned and copending U.S. application Ser. No. 09/614,243, filed Jul. 12, 2000, which claimed the benefit of commonly owned U.S. Provisional Application Ser. No. 60/151,901, filed Sep. 1, 1999.

### COPYRIGHT NOTICE

© Copyright 2000, 2003 John T. Cooker.

All rights reserved.

### BACKGROUND OF THE INVENTION

A portion of the disclosure of this patent document contains material that is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

1. Field of the Invention

The present invention generally relates to an oral delivery system.

2. Description of Related Art

The pharmaceutical industry has developed a variety of medications, medicaments, vitamins, nutritional supplements, etc. (collectively referred to herein as “oral dosages”) that can be taken orally and which are in the form of capsules, tablets, gel caps and their like (collectively referred to herein as “oral dosage forms”). However, many consumers have experienced difficulty in swallowing such oral dosage forms. Specifically, many types of commercially available oral dosage forms become buoyant on the liquid with which they are taken. This is especially the case with capsules which are hardened reservoirs of gelatin filled with powdered ingredients and air. As a result of such a configuration, the powdered ingredients and air become bubbles sealed in gelatin. The buoyancy of the oral dosage forms on the liquid causes discomfort and creates difficulty in swallowing. Specifically, the buoyancy characteristic creates the following problems:

1) the buoyancy of the oral dosage form works against the downward motion of swallowing and also reduces control of the oral dosage form by the tongue and pharynx muscles;

2) the dry gelatin outer surface of a capsule or gel cap, when wetted, quickly becomes sticky and easily adheres to surfaces it contacts. As a result, the oral dosage form may be left behind as it follows the liquid down the pharynx and esophagus thereby requiring successive swallows of additional liquid to flush down the oral dosage form; and

3) a capsule, while floating on the liquid, may move out of its intended aligned position in which the narrow end of its cylindrical shape points toward the pharynx and esophagus and as a result, is swallowed at an uncomfortable angle, possibly becoming lodged in the process.

Oral dosage forms that have a cylindrical, oval or rectangular shape (but not round), and which are not heavy enough to sink in the liquid with which they are taken may move out of their aligned position while being propelled by the tongue toward the pharynx and esophagus thereby being swallowed at an uncomfortable angle.

The pharmaceutical industry has attempted to solve these problems by developing various oral dosage forms that supposedly have improved swallowabilty. In their attempt to solve the aforementioned problem relating to swallowability, the pharmaceutical industry has focused on the size, shape and surface composition of the capsules, tablets, gel caps, etc. In another attempt to address the problem of swallowability, the industry developed and produced cylindrical-shaped tablets which were to replace round shaped tablets, (i.e. a capsule shaped tablet). One result of the pharmaceutical industry's attention to this problem was the development of the gelatin-coated caplets or tablets which not only addressed the problems relating to swallowability but also consumers' wariness of capsule tampering. Such a caplet is disclosed in U.S. Pat. No. 5,314,537. Although the gelatin-coated caplet has provided improvement in the swallowability of such caplets, the pharmaceutical industry's attempts to solve this problem are basically limited to the application of coatings to the exterior of the caplet, tablet, etc.

The prior art discloses several oral dosage forms, and drug delivery devices. Wong et al. U.S. Pat. No. 5,198,229 describes a fluid-imbibing drug delivery device having a first, low density such that it floats in the stomach contents for a predetermined prolonged period of time during which it dispenses a drug or other active agent to the stomach. This drug delivery device also has a second, higher density such that the device exits the stomach at the end of the predetermined prolonged period of time. The drug delivery device disclosed in Wong et al. has two chambers. One of these chambers is a buoyancy chamber. Due to the buoyancy chamber, the Wong et al. drug delivery device, in its first stage, floats on stomach fluids. The Wong et al. drug delivery device achieves density by release of air in the outer chamber. Therefore, the drug delivery device described in Wong et al. certainly cannot facilitate swallowing if it is configured to float first on liquids. Sharma et al. U.S. Pat. No. 4,894,233 discloses a drug delivery system comprising a core material comprising a drug, and a hydrophobic matrix coating the core. The coating delays hydration of the drug and masks the taste of the drug. The coating comprises an emulsifier, an edible fatty acid or wax and a glyceride. However, the use of fatty acids or waxes significantly increases the buoyancy of the drug delivery system. Such buoyancy can create difficulty in swallowing the drug delivery system. Eckenhoff U.S. Pat. No. 5,098,425 describes a ruminant dispensing device that comprises a density member or weight means that is dispersed in a hydrogel member. The dispensing device described in Eckenhoff is a time released dispenser which uses metals as densifying agents. Examples of such densifying agents described in Eckenhoff are iron, iron shot, iron shot coated with iron oxide, iron shot magnesium alloy, steel, stainless steel, copper oxide, a mixture of cobalt oxide and iron powder, a mixture of iron and copper oxide and the like. These metals are passed out of the animal's body. Such metal densifying agents or weight means are obviously not digestible and are clearly not suited for use by humans.

Despite the developments discussed above, consumers still continue to express desire for oral dosage forms that exhibit improved swallowability characteristics.

### SUMMARY OF THE INVENTION

It is therefore an object of the present invention to provide new and improved oral dosage forms that solve the aforementioned problems. Thus, the present invention is directed to improved oral dosage forms that are significantly easier to swallow. In accordance with the present invention, the oral dosage forms are configured to have relatively greater weight and/or density to effect partial or total submergence in the liquid with which the oral dosage form is taken. The oral dosage form has a size that conforms to a size that can be comfortably swallowed.

In one embodiment, the present invention is directed to an oral dosage form for ingestion with liquid, comprising, an active ingredient, an inactive ingredient, and a substance having a predetermined weight that effects at least partial sinking of the oral dosage form in the liquid. In a preferred embodiment, the substance is digestible. In one embodiment, the substance is a digestible filler material that is added to the active and inactive ingredients of the oral dosage form. In such an embodiment, the filler material is within the interior of the oral dosage form. In another embodiment, the filler material is added to the exterior of the oral dosage form. In a preferred embodiment, the filler material is digestible. In a preferred embodiment, the oral dosage form is configured so that at least the portion of the oral dosage form that has the filler (i.e. the weighted end of the oral dosage form) sinks below the surface of the liquid with which the oral dosage is taken.

In another embodiment of the invention, the substance is a binder that is used to increase the weight and/or density of the oral dosage form. In a preferred embodiment, the binder is digestible.

In accordance with one embodiment of the invention, the oral dosage form comprises a capsule that has a body portion and a cap portion, and the filler material is placed in the cap portion. In accordance with another embodiment of the invention, the oral dosage form comprises a capsule that has a body portion and a cap portion and the filler material is placed in the body portion. Other embodiments of the invention are possible as well. For example, a relatively heavier, denser or larger amount of active and inactive ingredients are used to formulate the oral dosage form. In another example, a combination of a binder and a relatively heavier, denser or larger amount of ingredient is used to formulate the oral dosage form.

In a preferred embodiment, the oral dosage form of the present invention does not utilize fatty acids, oils or waxes so as not to cause an increase in the buoyancy of the oral dosage form of the present invention. In a preferred embodiment, the oral dosage form of the present invention is configured to substantially eliminate the formation of any air pockets or air chambers so as to decrease the buoyancy of the oral dosage form.

Thus, in one aspect, the present invention is directed to an oral dosage form for swallowing with liquid, comprising an active ingredient, an inactive ingredient, and a digestible substance having a predetermined weight that effects at least partial sinking of the oral dosage form in the liquid. The active ingredient and inactive ingredient have a combined total weight that is substantially less than the predetermined weight of the digestible substance. In one embodiment, the oral dosage form comprises a capsule having a first end and a second end, and the active and inactive ingredients and the digestible substance are compressed within the capsule. The digestible substance is concentrated at the first end of the capsule so that when a user desires to ingest the capsule, the buoyancy of the capsule is substantially reduced and the capsule is aligned so that the first end of the capsule points in the direction of the pharynx and the second end of the capsule points in the direction of the mouth thereby substantially reducing the probability of the capsule being swallowed at an uncomfortable angle.

### BRIEF DESCRIPTION OF THE DRAWINGS

The features of the invention are believed to be novel. The figures are for illustration purposes only and are not drawn to scale. The invention itself, however, both as to organization and method of operation, may best be understood by reference to the detailed description which follows taken in conjunction with the accompanying drawings in which:

FIG. 1 is perspective view of a capsule configured in accordance with one embodiment of the present invention wherein a filler is added to the capsule.

FIG. 2 is a perspective view of a capsule in accordance with another embodiment wherein the capsule has indicia thereon to indicate the portion of the capsule having concentrated weight.

FIG. 3 is a perspective view of a capsule configured in accordance with a further embodiment of the present invention wherein filler granules are added to the capsule.

FIG. 4 is a perspective view of a capsule configured in accordance with yet another embodiment of the present invention wherein a single piece of filler is added to the capsule.

FIG. 5 is a perspective view of a capsule configured in accordance with a further embodiment of the present invention wherein the filler is attached or adhered to the exterior of the capsule.

FIG. 6 is a perspective view of a capsule configured in accordance with yet another embodiment of the present invention wherein the filler is molded into one of the capsule portions.

FIG. 7 is top plan view, partially in cross-section, of a tablet configured in accordance with one embodiment of the present invention wherein a tablet is embedded in a filler.

FIG. 8 is a top plan view, partially in cross-section, of a softgel configured in accordance with one embodiment of the present invention wherein a filler is embedded in one portion of the softgel.

FIG. 9 is a perspective view of a conical-shaped tablet configured in accordance with a further embodiment of the present invention wherein a filler substance is dispersed throughout the tablet.

FIG. 10 is a top plan view of a generally trapezoidal-shaped tablet having filler dispersed throughout the tablet.

FIGS. 11 and 12 are perspective views of a capsule in accordance with another embodiment of the present invention wherein a densifying substance is configured as a plug that is attached to one end of the capsule.

FIG. 13 is a perspective view of a capsule in accordance with another embodiment of the present invention wherein the filler is located in the cap portion of the capsule.

FIG. 14 is a table that shows typical commercially available capsule sizes and corresponding capsule volumes.

FIG. 15 is a table that shows specific filler weights, filler volumes and corresponding capsule sizes in accordance with the present invention.

FIG. 16 is a standardization table in accordance with the present invention.

FIG. 17 is a table that shows new capsule sizes, the amount of additional filler that can be added to the new capsules and the resulting total filler weight, in accordance with the present invention.

FIGS. 18A, 18B and 18C show a conversion table, in accordance with the present invention, which shows initial capsule sizes and various powder density and weights that can be achieved with each particular initial size capsule, and new capsule sizes and the various total densities and weights that can be achieved with each new capsule size.

### DETAILED DESCRIPTION OF THE INVENTION

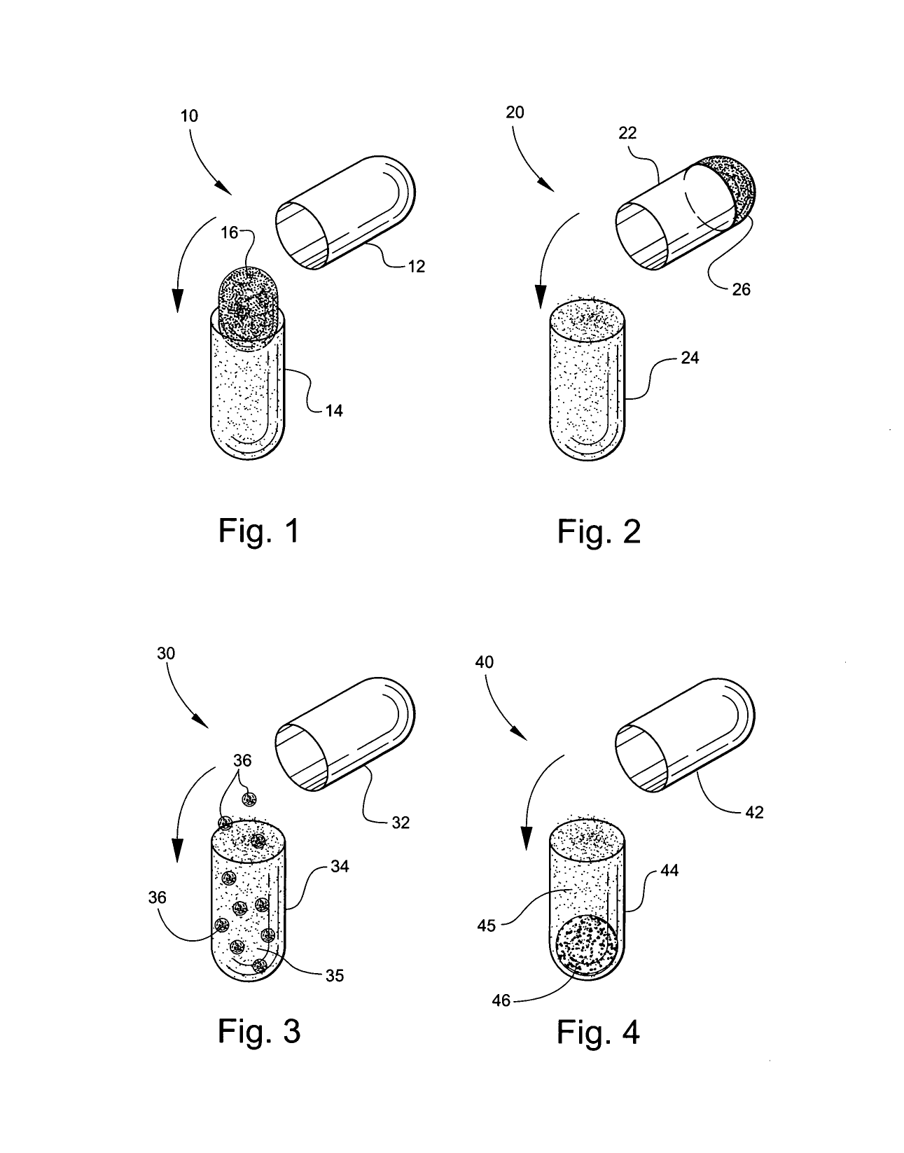

In describing the preferred embodiments of the present invention, reference will be made herein to FIGS. 1-18C of the drawings in which like numerals refer to like,features of the invention.

Oral dosage forms have a variety of physical characteristics. One such physical characteristic is the medium used for transport. Thus, the present invention teaches a variety of configurations of oral dosage forms and methods for making these oral dosage forms, that solve the aforementioned problems and deficiencies associated with conventional oral dosage forms. The configurations and methods of the present invention are applicable to the three major types of oral dosage forms: (1) capsules, (2) tablets and caplets, and (3) soft-gels.

It has been found that by increasing the weight of oral dosage forms, unexpected superior results are achieved in that the swallowability of the oral dosage forms is significantly improved. In particular, it has been found that an oral dosage form configured to have a relatively greater weight than that of a conventional, but same type, oral dosage form, has significantly improved swallowability characteristics. Specifically, the “weighted” oral dosage form results in a significantly greater gravitational force being exerted upon the oral dosage form which facilitates direct and relatively quicker passage of the oral dosage form through the pharynx and into the stomach.

It has also been found that increasing the density of oral dosage forms, unexpected superior results are achieved in that the swallowability of the oral dosage forms is significantly improved. In particular, it has been found that an oral dosage form configured to have a relatively greater density than that of a conventional, but same type, oral dosage form has significantly improved swallowability characteristics. Specifically, the oral dosage form having the greater density has significantly less buoyancy with respect to the liquid which is taken with the oral dosage form. As is described in the foregoing discussion, it is buoyancy that resists the swallowing process. Thus, by significantly reducing buoyancy of the oral dosage form, the swallowability of the oral dosage form is greatly enhanced.

It also has been found that increased weight and/or density concentrated at a particular end or side of an oral dosage form maintains the oral dosage form in a preferred and intended alignment by vertically positioning the oral dosage form as it is swallowed. The heavier end of the oral dosage form is positioned such that it points generally in the direction of the pharynx and the lighter end points generally toward the mouth. As a result, swallowability of the oral dosage form is significantly enhanced because the probability of the oral dosage form being swallowed at an uncomfortable angle is significantly reduced.

In accordance with one embodiment of the invention, the increased weight and/or density concentrated at a particular end or side of the oral dosage form of the present invention is such as to cause the oral dosage form to sink entirely in the liquid which is taken with the oral dosage form while maintaining the vertical alignment of the oral dosage form as discussed above. In accordance with another embodiment of the present invention, the increased weight and/or density concentrated at a particular end or side of the oral dosage form of the present invention is such as to cause the oral dosage form to partially sink in the liquid while maintaining the vertical alignment of the oral dosage form as discussed above.

As described above, in one embodiment of the present invention, a filler substance is added to the ingredients of the oral dosage form. The filler may be in solid, semi-solid, liquid or powder form. Furthermore, the filler may be inactive or may provide an active function, e.g. digestive aid. The filler can be formulated from any accepted pharmaceutical excipient materials, whether soluble or insoluble. Such excipient materials include sucrose, dextrose, lactose, fructose, microcrystaline cellulose, calcium carbonate, sorbitol, xylitol, isomalt, gelatin, and starches. However, it is to be understood that these aforementioned fillers are just examples and that other types of commercially available fillers can be used as well. The filler can be added at any convenient time during the filling process. In one embodiment, the filler material comprises solid sucrose granules that are added to the ingredients of an oral dosage form. In another embodiment, a solid sucrose portion is molded or adhered to the inside of an oral dosage form. In a further embodiment, the filler is applied as a relatively thick shell of sorbitol to the oral dosage form.

In accordance with one embodiment of the present invention, the filler is located in or at a particular portion of the oral dosage form so as to provide a weighted portion or a high-density portion. Thus, the weighted or high-density portion of the oral dosage form is relatively heavier than the remaining portion of the oral dosage form thereby causing the weighted or high-density portion to sink first in the liquid taken with which the oral dosage form is taken. The oral dosage form becomes vertically positioned within the liquid as a result of the weighted or high-density portion sinking first. In a preferred embodiment, the oral dosage form has indicia thereon to indicate which portion of the oral dosage form is the weighted portion. In the case of capsules, the indicia enables a consumer to align the weighted or heavier portion of the capsule in his or her mouth so as to use the capsule's cylindrical shape to facilitate correct alignment of the capsule and movement of the capsule through the throat and into the esophagus thereby reducing the chance that the oral dosage form will be swallowed at an angle that causes discomfort. As a result, confidence is instilled in the consumer when using the oral dosage form. In one embodiment, color is used as indicia to indicate the weighted portion of the oral dosage form.

In accordance with another embodiment of the invention, the oral dosage form may be configured such that the filler is part of the medium. In the case of a capsule, the filler is part of the capsule, either the body portion, cap portion, or both. In another embodiment, the filler is deposited on or disposed over the exterior surface of the medium of the oral dosage form. In one such embodiment, the filler is coated over the exterior surface of the medium of the oral dosage form. In another embodiment, a filler coating is applied to the exterior surface of the medium by dipping the oral dosage form in a liquid filler and then allowing sufficient time for curing and drying. In a preferred embodiment, the thickness of such a filler coating is between about 20 and 200 mils, inclusive.

In a further embodiment, a relatively heavier or denser diluent is used as an ingredient of the oral dosage form. In particular, a portion of a powdered diluent used to bulk the volume of a capsule containing a small dosage of ingredients is replaced with solid granules in order to increase the weight and/or density. In one example, rice flour in solid granule form is used. Rice flour in solid granule form has a relatively greater density or weight and as a result, can be used in the same available oral dosage form volume thereby providing a relatively heavier and denser oral dosage form.

In another embodiment, a binder is used to provide a relatively denser oral dosage form. Conventional capsules are typically filled with free flowing ingredients in powder form. However, conventional capsules do not use binders to adhere the free flowing ingredients. In accordance with another embodiment of the present invention, a binder is mixed with a portion of a capsule's ingredients in order to provide a significant increase in the density of the oral dosage form. In one embodiment, a starch binder is mixed with a portion of ingredients to form solids or semi-solids which, when dried and combined with the remaining ingredients, form a relatively high-density oral dosage form. As a result, a smaller capsule can be used, or a relatively larger amount of ingredients can be used in the original capsule. This process of binding a portion of the ingredients of a capsule requires relatively less effort than producing a complete tablet of all the ingredients and the resulting oral dosage form still retains the characteristics of a capsule, e.g. the capsule's shiny, smooth surface.

In another embodiment, relatively large amounts of diluent and binder are combined to form a composition that is used to formulate a tablet in order to increase the weight and/or density of the tablet. In another embodiment, a binder and a portion of an active ingredient or diluent of a capsule are mixed to form a solid which when dried, is added to the other portions of capsule ingredients thereby eliminating the need for a filler.

It is to be understood that any of the aforementioned methods and techniques of the present invention may be used in any combination to increase the weight and/or density of an oral dosage form.

It is to be understood that for purposes of facilitating understanding of the present invention, the ensuing description describes the liquid with which the oral dosage form is taken as water. However, it is to be understood that other liquids may be used, e.g. soda, juice, coffee, etc. and that the weight and/or density of the oral dosage form may have to be increased in accordance with the present invention to eliminate buoyancy of the oral dosage form on those types of liquids.

The instant specification describes inactive ingredients (e.g. excipients) that are used in the production of oral dosages. In a preferred embodiment, such excipients are commercially available accepted pharmaceutical excipient materials. In a most preferred embodiment, such excipients do not contain fatty acids or oils or waxes since such substances do increase buoyancy which is contrary to the objects of the present invention. In order to facilitate understanding of the present invention, the ensuing description is divided into separate discussions of each of the three types of oral dosage forms: (1) capsules, (2) tablets and caplets, and (3) soft-gels.

1) Capsules

As discussed in the foregoing description, conventional capsules, in effect, are sealed powder and air which have significant buoyant characteristics. In accordance with one embodiment of the present invention, a capsule is provided which is configured to have a weighted or high-density end portion. At least the weighted end portion of the capsule sinks below the surface of the liquid with which the capsule is taken. It has been found that sinking of at least the weighted end portion of the capsule in the liquid results in a significant improvement in the swallowability of the capsule without compromising other characteristics that consumers find favorable, e.g. shiny, smooth capsule surface. In accordance with this embodiment of the present invention, capsules are configured to have a relatively greater weight and/or density than water (the density of water is 1 g/ml @ 4° Celsius) without increasing the size of the capsule beyond consumer preference.

### EXAMPLE 1

A typical conventional capsule of 50 mg of the popular nutritional supplement zinc contains excipients that weigh 462 mg. Its total weight is 512 mg in a size 0 capsule with a volume of 0.68 ml and has a density D of 0.512 g/0.68 ml or 0.75 g/ml. Since the density of this capsule is less than 1 g/ml, it will float on water. In accordance with the present invention, the density D of the capsule is increased about 0.29 g/ml by the addition of a solid sucrose filler weighing 250 mg at 0.1 ml. As a result, the end product (i.e. the modified capsule) will weigh 762 mg at a volume of 0.73 ml. Due to compression in the filling process, the capsule will only expand 0.05 ml. Thus, the density-augmented capsule will now have a density D equal to 0.762 g/0.73 ml, or 1.04 g/ml. The density-augmented capsule will sink in the water with which it is swallowed thereby overcoming the problems associated with capsule buoyancy discussed in the foregoing description. Specifically, the density-augmented capsule will significantly reduce the probability of the capsule moving out of correct alignment and being swallowed at an uncomfortable angle. It is to be understood that the density of the density-augmented capsule can be further augmented. However, in a preferred embodiment, the density augmentation is such that it does not increase the size of the capsule beyond that which can be comfortably swallowed by consumers. However, it is to be understood that in the case of a veterinary application, particular animals may be able to swallow capsules having a size greater than the size preferred by consumers.

In Example 1 in the foregoing description, 250 mg or 32% of the total weight of the end product is the result of the addition of the solid sucrose filler. Since the range of capsule weight and density varies, the amount of the filler needed to achieve the objects of the present invention will also vary. In a preferred embodiment, the percentage of the total weight of the oral dosage form that is due to the weight of the digestible substance is between about 5% and 90%. In one embodiment, the percentage of the total weight of the end product that is due to the addition of the filler ranges between 15% and 80%, inclusive. In another embodiment, the percentage of the total weight of the end product that is due to the addition of the filler ranges between 25% and 50%, inclusive, if the filler is used exclusively.

### Example 2

In this example, the 512 mg weight of the conventional capsule described in Example 1 is significantly increased at the same volume by exchanging its powdered diluent for solid granules. Specifically, the 200 mg of rice flour power diluent is replaced by 400 mg of sucrose solid granules. The sucrose solid granules require the same amount of space but weigh twice as much as the rice flour diluent. Thus, the weight of the capsule is increased to 712 mg at the same volume with a density D of 0.712 g/0.68 ml or 1.04 g/ml. Thus, the weight-augmented capsule will sink in water.

### Example 3

In this particular example, a binder is used to produce a capsule having a relatively greater density. For this particular example, the conventional zinc capsule described above in Example 1 contains about 112 mg of powder zinc and other excipients and about 400 mg of the rice flour diluent. In this example, increasing the density of the capsule in accordance with the present invention comprises the following steps: (a) subtracting 200 mg of the rice flour diluent, (b) providing 200 mg of dry sorbitol, (c) mixing the sorbitol with liquid to form a syrup-like binder, (d) mixing the syrup-like binder with the remaining 200 mg of rice flour, (e) drying the mixture of the syrup-like binder and the rice flour, (f) forming granules from the dried mixture, and (g) adding the granules to the 112 mg of powder zinc and remaining excipients. As a result, the total capsule weight remains about the same but the volume of the capsule ingredients (zinc, excipients and binder) shrinks or decreases 0.2 ml allowing the use of smaller capsule portions. The density D of the end product will be 0.512 g/0.5 ml or 1.024 g/ml. This density is more than sufficient to allow the capsule to sink in water. If water is to be used as the liquid medium with which the capsule is taken, then it is preferable that the density of the capsule be increased to a density that is greater than 1 g/ml so as to effect sinking of substantially the entire capsule in the water. However, it is to be understood that the density of the capsule, or of any of the oral dosage forms, can be increased in accordance with the present invention to effect sinking of the oral dosage form in other types of liquids, e.g. soda, juices, coffee, milk, etc.

In an alternate embodiment of the present invention, filler is placed in the body portion of the capsule along with the ingredients. The cap portion is then attached to the body portion. In such a configuration, the size of the body portion is increased (e.g. elongated) so as to enable filler and ingredients to fit in the body portion. In accordance with such an alternate embodiment, a solid filler of micro-crystalline cellulose or lactose is inserted into the body portion before the empty capsules are shipped. In a preferred embodiment, adhering agents are used. In order to achieve maximum effect, a maximum weight and volume of filler should be used with a given capsule size thereby creating a system of standardization of filler sizes and corresponding increased capsule sizes.

The following example illustrates the embodiment of the present invention wherein the filler is placed or positioned in the cap portion of the capsule.

### EXAMPLE 4

A typical conventional capsule of 50 mg of the popular nutritional supplement zinc contains excipients that weigh 462 mg. Its total weight is 512 mg in a size 0 capsule with a volume of 0.68 ml and has a density D of 0.512 g/0.68 ml or 0.75 g/ml. Since the density of this capsule is less than 1 g/ml, it will float on water. In accordance with the present invention, the density D of the cap portion of the capsule is increased from approximately zero, when it is empty, to about 2.5 g/ml by the addition of a solid sucrose filler weighing 250 mg at 0.1 ml. As a result, the cap portion of the end product (i.e. the modified capsule) will sink below the surface of the water with which the capsule is ingested thereby overcoming the problems associated with capsule buoyancy discussed in the foregoing description. Specifically, as a result of the cap portion sinking in the water, the density-augmented capsule properly aligns itself such that the whole capsule is parallel with the esophagus thereby preventing the capsule from being swallowed at an uncomfortable angle. Furthermore, the density-augmented capsule travels directly down the esophagus and is not left behind to stick to the esophagus surface.

### EXAMPLE 5

A 0.500 ml size #1 capsule is typically used to fill 400 mg of ingredients. In accordance with this embodiment of the present invention, the size of the capsule is increased to a size #00 with a 0.950 ml capacity. The additional 0.450 ml of volume is used for the filler. When a filler having a density of 1.5 g/ml is used, the extra 0.450 ml volume would accommodate a filler weighing 675 mg. Thus, standardization can be achieved for any size capsule.

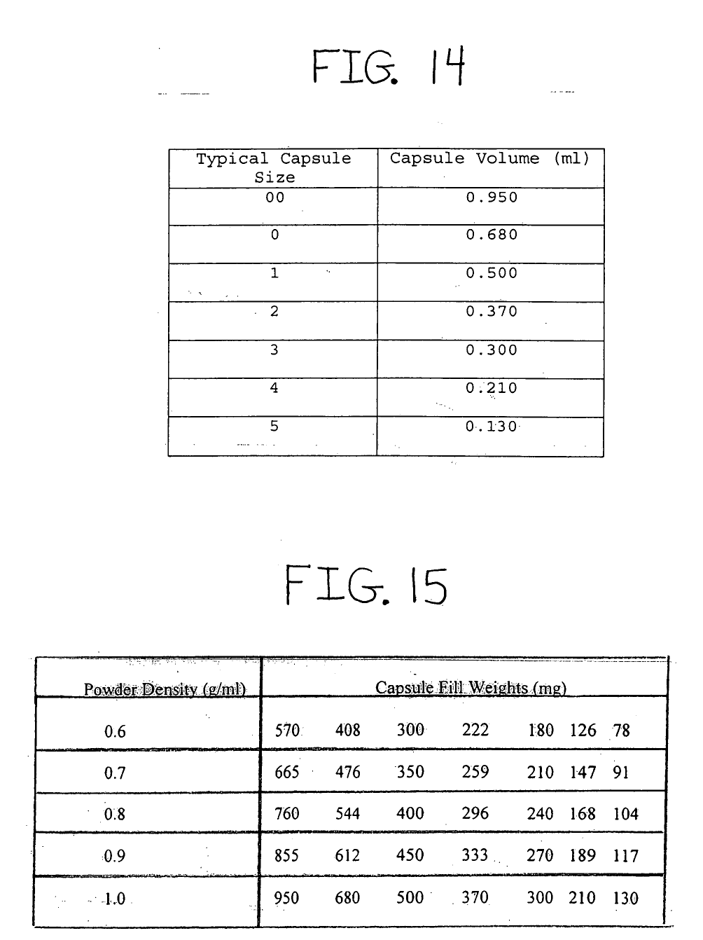

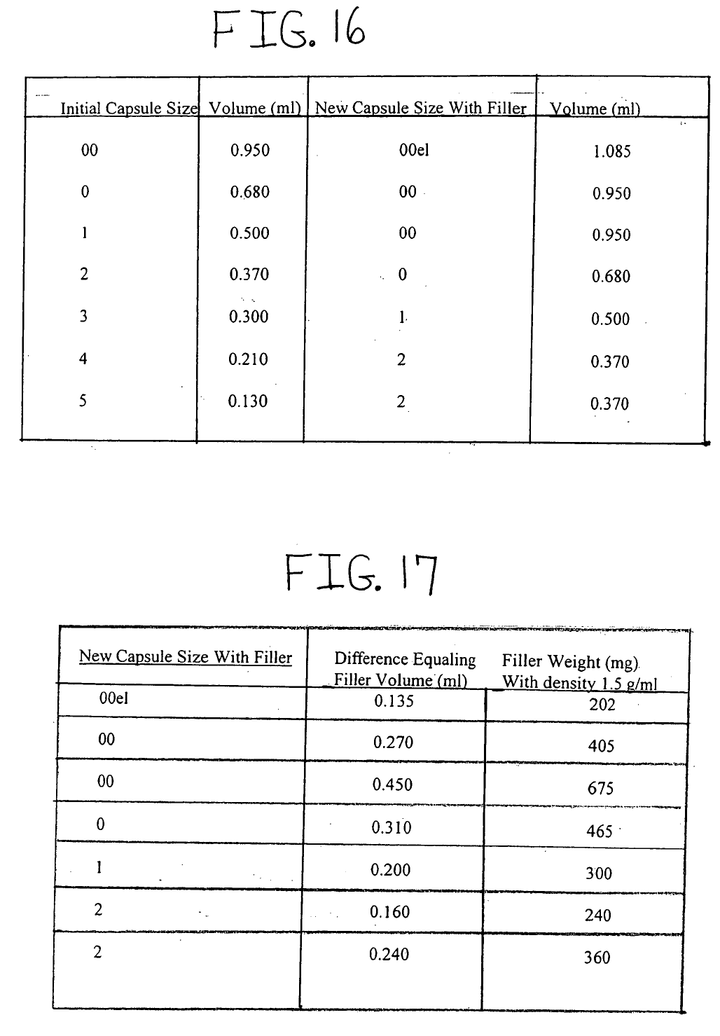

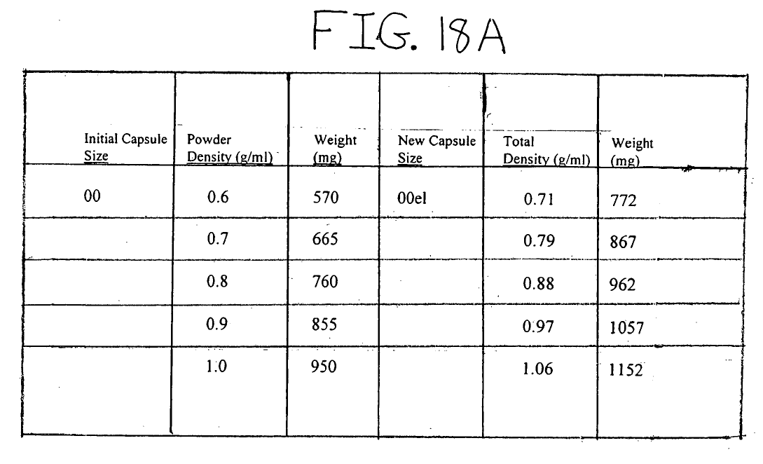

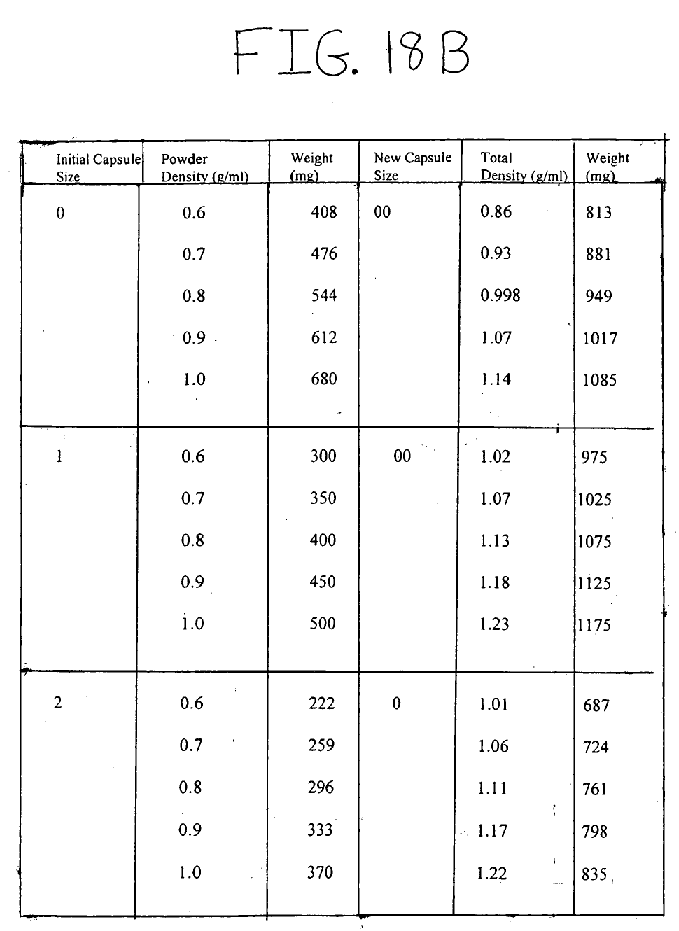

In another embodiment of the present invention, a size #00 capsule is configured to have an elongated geometry so as to provide a further 0.135 ml of volume or 14% increase in space) for a filler weighing 202 mg. As used herein, the designation “el” refers to such elongated capsule geometry. Referring to FIG. 14, there is shown a table that shows typical commercially available capsule sizes and corresponding capsule volumes. Referring to FIG. 15, there is shown a table that shows specific filler weights, filler volumes and corresponding increased capsule sizes in accordance with the present invention. The additional filler increases the density of the finished capsule to a density that is substantially close to or above the density of water. Lactose or micro-crystalline fillers have a density of 1.5 in solid form. Thus, in accordance with the present invention, FIG. 16 shows a standardization table for this type of filler (i.e. having a density of 1.5 in solid form) for each respective capsule size. Referring to FIG. 17, there is shown a table that shows new capsule sizes, and the amount of additional filler that can be added to the new sized capsules and the resulting total filler weight. As shown in FIGS. 16 and 17, capsule sizes 4 and 5 are increased to size 2. The difference in a size 5 capsule and a size 2 capsule is 0.24 ml which will accommodate a filler weight of 360 mg (0.24 ml×1.5 mg (solid filler density)). The difference in a size 4 capsule and a size 2 capsule is 0.16 ml which can accommodate a filler weight of 0.16 ml×1.5 mg=240 mg.

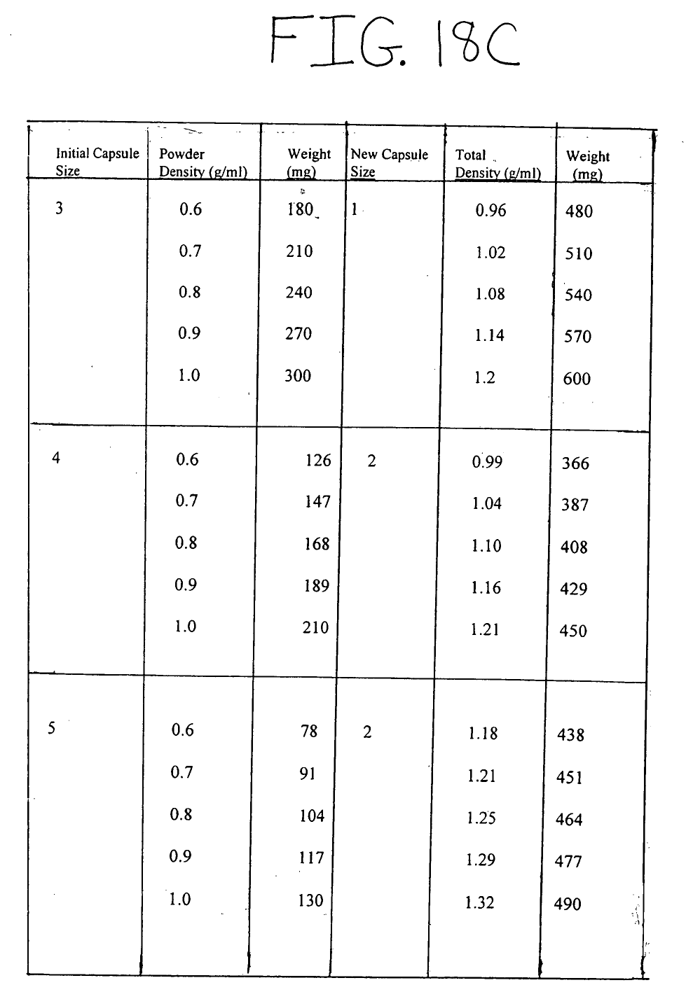

Referring to FIGS. 18A, 18B and 18C, in accordance with the present invention, there is shown a conversion table that shows initial capsule sizes and various powder density and weights that can be achieved with each particular initial size capsule, and new capsule sizes and the various total densities and weights that can be achieved with each new capsule size. As shown in FIGS. 18A, 18B, and 18C, the various total densities and weights that can be achieved with each new capsule size are relatively greater than the densities and weights that are possible for the corresponding initial capsule sizes. Thus, the table of FIGS. 18A, 18B, and 18C provides a standardization system which provides seven (7) capsule sizes that accommodate the standard capsule sizes at four or more powder densities. Thus, the increase in densities is clearly demonstrated.

In order to produce the oral dosage forms in accordance with the present invention, particular, pertinent and novel manufacturing steps are implemented. These steps are described in the ensuing description.

Capsules typically are comprised of two portions (i.e. body portion and cap portion) that are pushed together to enclose the capsule ingredients. The capsule portions are pushed together by applying a predetermined force. This type of “enclosing” technique does not employ any steps to seal the capsule portions together after the predetermined force is applied. However, it has been found that the force currently being used in conventional processes to compress the ingredients to an effective density is not sufficient because the capsule ingredients tend to bounce back when the capsule portions are not sealed together. The method of the present invention includes a particular step that pertains to the application of such force. Specifically, the method of the present invention includes the step of applying a relatively greater force to the capsule portions, in comparison to the force applied in conventional capsule fabrication processes, in order to extract substantially all of the air from the powdered ingredients. The extraction of air from the interior of the capsule facilitates swallowability of the capsule. Furthermore, the increased force facilitates the positioning of the filler and the ingredients within the capsule volume.

In another embodiment of the method of the present invention, the capsule portion holding the ingredients is configured to have a deeper well to accommodate the filler material. This is illustrated in FIG. 1. Capsule 10 generally comprises cap portion 12 and body portion 14. Body portion 14 holds the ingredients during the filling process. In one embodiment, body portion 14 is configured to have a relatively deeper “well” in order to accommodate filler material or a binder that is used to increase the weight and/or density of the capsule in accordance with the present invention. In such an embodiment, the length of body portion 14 is increased or elongated to accommodate filler material or binders. In another embodiment, the length of cap portion 12 is increased or elongated to accommodate filler material or binders. In yet a further embodiment, the diameter of portions 12 and 14 are increased to accommodate larger amounts of filler materials or binders.

The filler may be configured to have any suitable shape. However, it has been found that if a single piece of solid filler is used in a capsule, then the preferred shape of the solid filler should be generally spherical or oval in order to facilitate compacting powder ingredients and filling air gaps. In one embodiment, the filler is covered with a film material that prevents it from interacting with the ingredients.

In a further embodiment of the method of the present invention, the capsule components, e.g. portions 12 and 14, are formulated to have a relatively greater thickness to provide relatively greater strength to withstand the greater force resulting from addition of the filler. Furthermore, it is to be understood that the added thickness of the capsule portions also contributes to the increase in weight and density of the capsule in accordance with the present invention.

In another embodiment of the method of the present invention, a sealing process is used to hold the joined capsule portions in place. Specifically, this sealing process comprises the step of applying an adhering agent around the seam of the filled and joined capsule portions.

In a further embodiment, a locking process is used to hold the joined capsule portions in place. In such a process, indentations or moldings are formed on the capsule cap and body. The indentations or moldings are used to lock the cap and body together. A plurality of indentations or moldings can be used to provide capsules of varying lengths.

In yet another embodiment of the method of the present invention, when the weight of the capsule is increased to improve swallowability, as described above, the weight is concentrated at one end of the capsule. When such a configuration is used, markings or indicia are applied to the weighted portion of the capsule so as to indicate the weighted portion of the capsule. Such a configuration is shown in FIG. 2. Capsule 20 comprises cap portion 22 and body portion 24. Portion 22 has indicia 26 to indicate that portion 22 is the weighted portion and that capsule 20 should placed in the consumer's mouth so that weighted portion 22 is pointing toward the opening of the esophagus. In one embodiment, the indicia 26 consist of a color. However, it is to be understood that other types of indicia can be used, e.g. letters, numbers, etc.

Referring to FIG. 3, as described in the foregoing description, granules of a particular substance, e.g. sucrose solid granules, are added to the capsule to increase the weight and/or density of the capsule. Thus, in this embodiment of the method of the present invention, the method comprise the steps of (a) providing capsule 30 that comprises capsule portions 32 and 34, (b) retaining portion 34 so that it is stationary, (c) depositing ingredients 35 into portion 34, (d) depositing granules 36 into portion 34, and (e) joining capsule portions 32 and 34 together. In another embodiment, this particular method includes the step of applying indicia to the weighted portion of capsule 30 in a manner described above.

Referring to FIG. 4, the method of the present invention also provides particular steps for using a single piece of filler substance. Specifically, these particular steps comprise (a) providing capsule 40 that comprises cap portion 42 and body portion 44, (b) retaining body portion 44 so that it is stationary, (c) depositing ingredients 45 into body portion 44, (d) depositing single filler substance 46 into body portion 44, and (e) joining cap portion 42 to body portion 44. In one embodiment, single filler substance 46 is deposited into body portion 44 by capsule filling machines known in the industry. In one embodiment, this method includes the step of applying indicia to the weighted portion of capsule 40 in a manner as described above. In one embodiment, the predetermined weight and concentration of the filler in portion 44 produces a density of portion 44 that is at least 1.0 g/ml.

In a further embodiment of the present invention, a capsule is configured to have a body portion (e.g. portion 44) that is sized to contain the active ingredients, any required inactive ingredients, and the filler, and a flat cap portion (not shown) that is used to seal the body portion thereby substantially eliminating any air pockets or air reservoir.

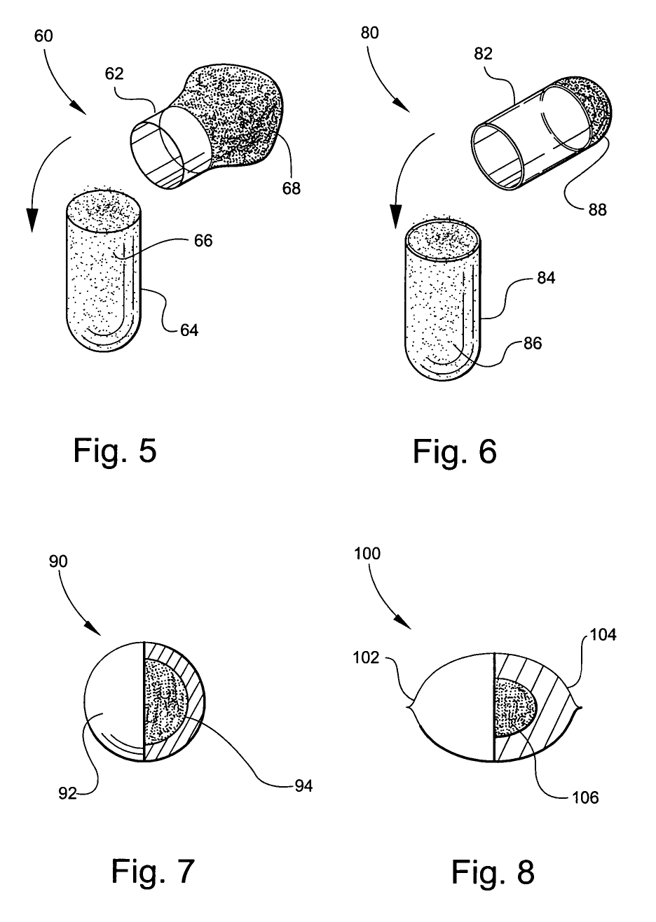

Referring to FIG. 5, in another embodiment of the method of the present invention, a method is provided for adding or attaching a filler to the exterior of either capsule portion. Specifically, this method comprises the steps of (a) providing capsule 60 that comprises capsule portions 62 and 64, (b) retaining portion 64 so that it is stationary, (c) depositing ingredients 66 into portion 64, (d) adhering or attaching filler substance 68 to the exterior surface of portion 62; and (e) joining capsule portions 62 and 64 together.

Referring to FIG. 6, in a further embodiment of the method of the present invention, a method is provided for molding a filler substance into either of the capsule portions. Specifically, this method comprises the steps of (a) providing capsule 80 that comprises capsule portions 82 and 84, (b) retaining portion 84 so that it is stationary, (c) depositing ingredients 86 into portion 84, (d) molding filler substance 88 into portion 82, and (e) joining capsule portions 82 and 84 together. In an alternate embodiment of this method, the filler substance 88 is adhered to the inside of either of the capsule portions. In another embodiment, the body portion or cap portion, or both, are molded to have a portion comprised of the filler substance. In a further embodiment of the present invention, one or both of the capsule portions are formed from the filler substance. Thus, in such an embodiment, the entire capsule, i.e. both body and cap portions, are made from the filler substance.

Referring to FIG. 13, there is shown a further embodiment of the present invention. Density-augmented capsule 400 comprises body portion 402 having an interior for containing ingredients 403 (e.g. active ingredients and any inactive ingredients), and cap portion 404 having an interior 405 for receiving filler substance 406 (shown in phantom). Filler 406 can be in solid, semi-solid or paste form. Preferably, filler substance is digestible. Thus, in accordance with the invention and the embodiment shown in FIG. 13, the following method is provided (a) providing capsule 400 that comprises capsule body portion 402 and cap portion 404 which has interior 405, (b) retaining body portion 402 so that it is stationary, (c) depositing ingredients 403 into the interior of body portion 402, (d) depositing filler substance 406 into interior 405 of cap portion 404, and (e) joining cap portion 404 to body portion 402.

In another embodiment of the present invention, the solid or semi-solid filler is placed or positioned only in the interior of the body portion of the capsule. In a further embodiment, solid or semi-solid filler is placed in both the interiors of the cap and body portions of the capsule.

In yet another embodiment, a capsule is configured to comprise a body portion for containing the active ingredients and a plug portion. The plug portion is formed from a filler substance. The filler substance can be in solid form or paste form. This embodiment is shown in FIGS. 11 and 12. Capsule 300 comprises body portion 302 and plug portion 304. Body portion 302 has an opening 306 and contains the active ingredients. Plug portion 304 is formed from the filler and is inserted into opening 306. The size of plug portion 304 is chosen so as to create a frictional fit between the plug portion 304 and body portion 302. The amount of active ingredients and the size of plug portion 304 are chosen so as to substantially eliminate air pockets within capsule 300. In a further embodiment, body portion 302 is elongated or lengthened, in comparison to conventional capsule body portions, so as to accommodate the active ingredients and plug portion 304. Plug portion 304 can be plugged or inserted into body portion 302 by mechanical means such as typical capsule filling machines which are known in the industry. An advantage of capsule 300 is that plug portion 304 renders a typical gelatin cap unnecessary.

The solid filler described above can be standardized in order to facilitate automated manufacturing processes. The solid filler can be adhered to the inside of the cap portion of the capsule while the cap portion is still empty. Capsules typically are manufactured in standard sizes so that they may be handled by automatic filling machines.

The feature of concentrating the filler at one end of the capsule provides many advantages. One such advantage is that the end of the capsule having the filler becomes submerged in the liquid even if the total density of the capsule does not exceed that of water. This advantage results from the proportionally larger weight of the “filler end” of the capsule. Even if the lighter end of the capsule having the powder ingredients still floats, the effectiveness of the capsule of the present invention is still achieved by the weighted filler end of the capsule being submerged. Another advantage is the improved swallowability of the capsule. A further advantage is that the proportionally larger weight of the filler end of the capsule counteracts the buoyancy caused by air bubbles that may be created when the cap portion of the capsule is joined with the body portion.

In a further embodiment, the cap portion of the capsule is configured so as to substantially degrade the formation of any bubbles therein when the cap portion of the capsule is joined with the body portion. In one embodiment, this is achieved by configuring the end of the cap portion to have a generally flat geometry rather than a curved or rounded end. In another embodiment, the interior portion of the cap portion that is adjacent to the end of the cap portion is filled in with the same material used to formulate the cap portion.

2) Tablets and Caplets

In accordance with the present invention, tablets and caplets are produced with relatively greater weight and density to improve swallowability of the tablets and/or caplets. The increase in weight and density is accomplished by adding a filler as described above. In one embodiment, the filler is added to the ingredients. For example, in one embodiment, a solid sucrose filler is molded in with the ingredients. In another example, the filler is embedded within the tablet ingredients.

In another embodiment, the tablet or caplet is configured such that the active ingredients and any inactive ingredients are embedded in a filler substance. This example is illustrated in FIG. 7. Tablet 90 comprises portions 92 and 94. Portion 92 is comprised only of filler. Portion 94 is embedded within the filler. Portion 94 comprises the active ingredients and any inactive ingredients.

In yet another embodiment, the tablet is configured such that one side, end or portion of the tablet has concentrated weight and has indicia to indicate which side, end or portion has the concentrated weight.

In a further embodiment, increasing the weight and density of the tablet or caplet is accomplished by increasing a component of the tablet or caplet. In one embodiment, this is accomplished by increasing the weight of an excipient such as the binder ingredient.

In a preferred embodiment, the percentage of the total weight of the end product that is due to the addition of the filler, or the increase in the weight of the component, is between about 15% and 90%, inclusive. More preferably, the percentage of the total weight of the end product due to the addition of the filler or increase in the weight of the component is between about 25% and 75%, inclusive. For example, 50% of the weight of a 400 mg tablet augmented with 200 mg of filler is the result of adding the filler. In this example, the original weight of the tablet has been doubled or increased about 100%.

The increase in weight and/or density provides significantly improved swallowability of particularly light, small sized tablets and caplets (i.e. under 300 mg, 4/10 ml).

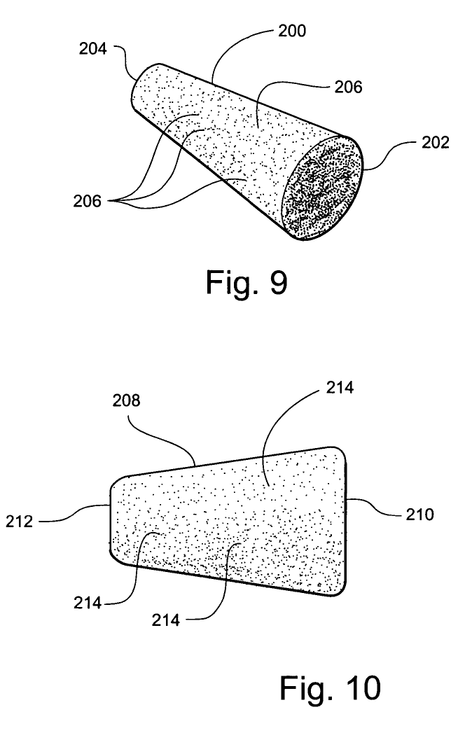

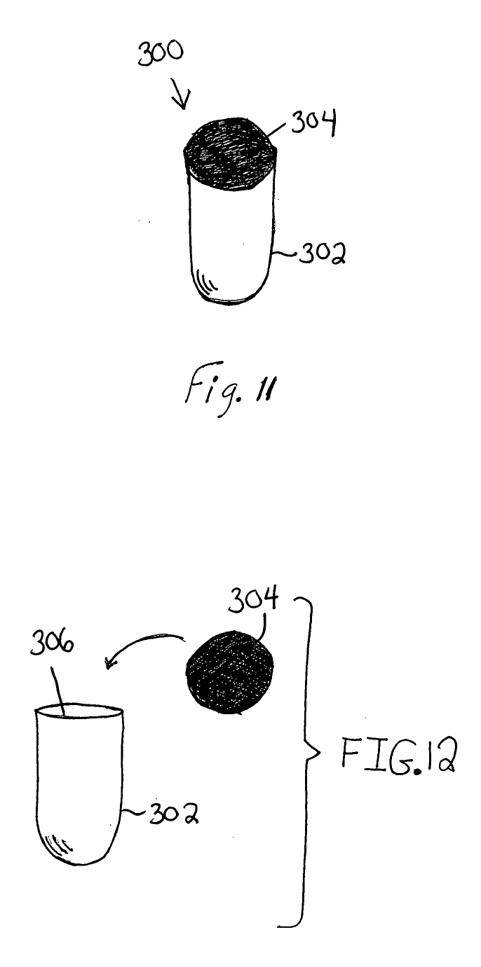

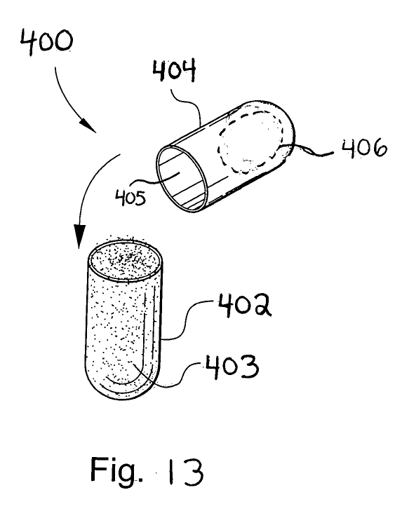

Referring to FIGS. 9 and 10, there are shown alternate embodiments of the tablets and caplets of the present invention. These embodiments utilize a particular shape having a swelled end which is substantially larger than the opposite end. For example, FIG. 9 shows a conical-shaped tablet or caplet 200 which has swelled or enlarged end 202 and a relatively narrow end 204. Filler 206 is dispersed throughout tablet or caplet 200. FIG. 10 shows a generally-trapezoidal shaped tablet or caplet 208 which has a swelled or enlarged end 210 and a relatively narrow end 212. Similarly, filler 214 is dispersed throughout the tablet or caplet 208. As a result of the shape of tablets or caplets 200 and 208, swelled ends 202 and 210, respectively, are substantially heavier than the opposite narrow ends. Thus, the swelled or enlarged ends sink in the liquid with which the tablet or caplet is taken thereby effecting correct alignment of the tablet or caplet and movement of the tablet or caplet through the throat and into the esophagus.

3) Softgels

In accordance with the present invention, the weight of softgels is increased to improve swallowability thereof. In one embodiment of the present invention, increasing the weight of softgels is accomplished by the addition of a filler substance. Specifically, the filler is added to the ingredients, e.g. a solid sucrose portion. This is illustrated in FIG. 8. Softgel 100 comprises portions 102 and 104. Portion 102 is comprised only of ingredients. Portion 104 includes filler 106. In one embodiment, filler 106 is comprised of solid sucrose.

In another embodiment of the present invention, increasing the weight of softgels is accomplished by increasing the amount of a component. For example, if the component is a liquid excipient, then the amount of the excipient is increased. Increasing the amount of the component can also be realized by increasing the size or thickness of the softgel.

In another embodiment, the softgels can be configured so that one side or portion of the softgel has concentrated weight and has indicia to indicate this end.

In a preferred embodiment, the percentage of total weight of the softgel that is due to the addition of the filler or increase in component is between about 15% and 90%, inclusive. More preferably, the percentage of total weight of the softgel that is due to the addition of the filler or increase in component is between about 25% and 75%, inclusive.

The increase in weight and/or density provides significantly improved swallowability of particularly light, small sized softgels (i.e. under 300 mg, 4/10 ml).

Gravity plays a significant role in the process of swallowing food. The role of gravity in swallowing food is described in The Human Body, Volume on “Digestion”, pp. 60-61, Torstar Books, 1984 and in The ABC's of the Human Body, Reader's Digest General Books, page 240, 1987. The relatively greater weight and/or density of the oral dosage forms of the present invention effect augmentation of the gravitational force that facilitates the downward passage of the oral dosage forms. Thus, the swallowability of the oral dosage forms is significantly increased.

Thus, the present invention provides new and improved oral dosage forms that:

a) solve the aforementioned problems discussed above that relate to the swallowability of oral dosage forms;

b) can be provided in the form of capsules, tablets, gelcaps and softgels;

c) interact with gravity to facilitate prompt and direct movement of the oral dosage form to the opening of and through the esophagus;

d) results in a relatively faster dissolution rate of the oral dosage form and relatively faster absorption rate of the medication provided by the oral dosage form;

e) can be produced with commercially available ingredients; and

f) can be produced without exorbitant manufacturing costs.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations in changes may be made by those skilled in the art without departing from the spirit of the invention. Accordingly, the foregoing detailed description should be considered exemplary in nature and not limited to the scope and spirit of the invention as set forth in the attached claims.

Thus, having described the invention, what is claimed is:

1. An oral dosage form for swallowing with liquid, comprising:
an active ingredient;
an inactive ingredient; and
a digestible substance having a predetermined weight that effects at least partial sinking of the oral dosage form in the liquid.

2. The oral dosage form according to claim 1 wherein the active ingredient and inactive ingredient have a combined total weight that is substantially less than the predetermined weight of the digestible substance.

3. The oral dosage form according to claim 1 wherein the percentage of the total weight of the oral dosage form that is due to the weight of the digestible substance is between about 5% and 95%.

4. The oral dosage form according to claim 1 wherein the digestible substance is a filler.

5. The oral dosage form according to claim 4 wherein the filler is a solid.

6. The oral dosage form according to claim 4 wherein the filler is a semi-solid.

7. The oral dosage form according to claim 4 wherein the filler is coated over the active and inactive ingredients.

8. The oral dosage form according to claim 1 wherein the digestible substance is a binder.

9. The oral dosage form according to claim 1 wherein the oral dosage form comprises a capsule having a first end and a second end, and wherein the active and inactive ingredients and the substance are compressed within the capsule.

10. The oral dosage form according to claim 9 wherein the digestible substance is concentrated at the first end of the capsule.

11. The oral dosage form according to claim 10 wherein the predetermined weight effects sinking of substantially the entire first end of the capsule in the liquid.

12. The oral dosage form according to claim 10 wherein the capsule has indicia thereon to indicate the first end of the capsule.

13. The oral dosage form according to claim 9 wherein the digestible substance is a filler which is molded to the inside of the capsule.

14. The oral dosage form according to claim 9 wherein the digestible substance is a filler which is adhered to the inside of the capsule.

15. The oral dosage form according to claim 1 wherein the active and inactive ingredients and the digestible substance are configured to form a tablet having a first portion comprising the active ingredient and a second portion comprising the digestible substance.

16. The oral dosage form according to claim 1 wherein the active and inactive ingredients and the digestible substance are configured to form a caplet having a first portion comprising the active and inactive ingredients and a second portion comprising the digestible substance.

17. The oral dosage form according to claim 1 wherein the percentage of the total weight of the oral dosage form that is due to the weight of the digestible substance is between about 5% and 95%.

18. An oral dosage form for ingestion with liquid, comprising:
an active ingredient;
an inactive ingredient; and
a digestible substance having a predetermined weight that effects an increase in the density of the oral dosage form to at least 1.0 g/ml.

19. The oral dosage form according to claim 18 wherein the percentage of the total weight of the oral dosage form that is due to the weight of the digestible substance is between about 5% and 95%.

20. The oral dosage form according to claim 19 wherein the percentage of the total weight of the oral dosage form that is due to the weight of the digestible substance is between about 15% and 80%.

21. An oral dosage form configured to be swallowed in whole with a liquid, comprising:
a capsule having a first portion and a second portion attached to the first portion, each portion having an interior;
an active ingredient concentrated in the interior of the first portion; and
a predetermined amount of digestible filler concentrated in the interior of the second portion of the capsule, the filler being chosen from the group sucrose, dextrose, lactose, fructose, microcrystaline cellulose, sorbitol, xylitol, isomalt, gelatin and starch, the filler having a predetermined weight that increases the density of the capsule to a predetermined density that effects sinking of at least the second portion of the capsule below the surface of the liquid, the amount and concentration of the filler being such as to eliminate formation of regions within the interior of the second portion that are relatively low density with respect to the liquid with which the capsule is swallowed;
wherein the percentage of the total weight of the capsule that is due to the weight of the filler is between about 5% and 95% and wherein the amount and concentration of the filler in the interior of the second portion significantly reduce the buoyancy of the capsule so as to cause at least the second portion of the capsule to sink below the surface of the liquid.

22. The oral dosage form according to claim 21 wherein the first portion of the capsule comprises a body portion of the capsule and the second portion comprises a cap portion of the capsule.

23. The oral dosage form according to claim 21 wherein the predetermined weight and concentration of the filler in the second portion produces a density of the second portion that is at least 1.0 g/ml.

24. An oral dosage form configured to be swallowed in whole with a liquid, comprising:
a body portion having an opening and an interior;
an active ingredient concentrated in the interior of the body portion;
ingredients disposed in the interior of the body portion; and
a plug portion attached to the body portion so as to seal the opening, the plug portion comprising a predetermined amount of digestible filler; and wherein the percentage of the total weight of the oral dosage form that is due to the weight of the filler is between about 5% and 95% and wherein the amount and concentration of the filler significantly reduce the buoyancy of the oral dosage form so as to cause a significant portion of the oral dosage form to sink below the surface of the liquid.

25. A method for making a density-augmented capsule, comprising:
providing a capsule body portion having an interior for receiving ingredients;
providing a capsule cap portion that has an interior for receiving a filler substance;
retaining the capsule body portion so that it is substantially stationary;
depositing ingredients into the capsule body portion; depositing a filler substance into the cap portion; and
joining the capsule cap portion to capsule body portion.

26. The method according to claim 25 wherein the ingredients include active ingredients.

27. The method according to claim 26 wherein the ingredients further include inactive ingredients.

28. A tablet for ingestion with a liquid, comprising:
a first end;
a second end;
an active ingredient;
an inactive ingredient; and
a digestible substance having a predetermined density which is concentrated at the first end so as to effect sinking of at least a portion of the tablet in the liquid.

29. The oral dosage form according to claim 28 wherein the percentage of the total weight of the tablet that is due to the weight of the digestible substance is between about 15% and 80%.

30. The table according to claim 28 wherein the tablet has a generally conical shape which tapers from the first end to the second end.

31. The table according to claim 28 wherein the tablet has a generally trapezoidal shape which tapers from the first end to the second end.
